(19) United States
(12) Patent Application Publication
(10) Pub. No.: US 2023/0102574 A1
(43) Pub. Date: Mar. 30, 2023

(54) DEVICE TO DEFINE THE RETENTION BOUNDARY OF GRANULAR MATERIALS

(71) Applicant: Richard E. Nicholson, Eagleville, PA (US)

(72) Inventor: Richard E. Nicholson, Eagleville, PA (US)

(21) Appl. No.: 17/485,473

(22) Filed: Sep. 26, 2021

(51) Int. Cl. G01N 11/02; G01N 33/00

(52) U.S. Cl. CPC G01N 11/02; G01N 33/00; G01N 2033/0091

(57) ABSTRACT

A device and test method for characterizing granular materials for angle of repose, static and dynamic flow properties, and process parameter variables by means of defining a sample retention boundary. The device is composed of a funnel for dynamic testing, a retention ring for static testing, a base, and a test device having an upper surface set at one or more angles to determine conditions in which samples are retained. By varying the texture of the surface, flow characteristics and other process issues can be evaluated.

### TECHNICAL FIELD

The present disclosure relates to, in general, test devices for characterizing dry powders, blends, mixtures, and granular materials for flow properties.

### BACKGROUND

Angle of repose has been used in several industries to characterize the flow properties of dry powders, granulations, and aggregates. According to the United States Pharmacopeia, “Angle of repose is a characteristic related to interparticulate friction or resistance to movement between particles” and is therefore used to predict manufacturing performance of dry powders or granular materials. A granular material loaded onto a flat surface will form a conical pile. The steepest angle in which the material can be piled without slumping is known as the angle of repose. Round dense uniformly sized granular materials exhibiting excellent flow properties will form shorter piles and therefore have a lower angle of repose. Granules having non-uniform surfaces, morphologies, densities, or particle sizes, or are susceptible to static build up typically have poor flow properties and form taller piles with a higher angle of repose. Since bulk material handling and manufacturing operations require specific flow properties to achieve the desired results, tests have been developed as a means of assuring product performance.

Angle of repose measurement as described in the U.S. Pharmacopeia Section <1174> Powder Flow as shown in FIG.1 is commonly utilized within the pharmaceutical, chemical, and food industries. The apparatus consists of an adjustable stand set on a flat level surface 110. The granular material 116 flows onto a base 126 of fixed diameter upon opening a gate 118 located at the bottom of the funnel 114. Upon initiating the test, granular material pours onto a base 126 consisting of a level round surface with a lip around the outer edge to aid in the formation of the conical pile. Material will continue to pile upon the base 126 until an excess of material spills over the sides at which point flow is halted. A uniformly shaped conical pile 112 is ideally formed at an angle proportional to flow characteristics of the granular material. The angle formed between the upper surface of the conical pile 122 and the horizontal surface 124 is known as angle of repose (α) and can be is measured directly using a protractor, or after measuring the height of the pile, calculated using the equation: tan(α)=2h/d; where d is the diameter of the conical pile at its base and h is the height of the conical pile. Issues associated with this method include error due to the formation of a non-uniform cone.

### SUMMARY OF THE INVENTION

The invention consists of novel test devices having a variably angled surfaces topography to characterize angle of repose and other flow properties of powders and granular materials using a simplified procedure. A test sample is poured, by hand or preferably through a fixed funnel, onto the surface of a device to determine under which conditions a conical pile will form. Since materials will only form a conical pile upon surfaces at angles below or equal to its own angle of repose, the angle of repose angle can therefore be determined. Test device designs may have a single angle, multiple angles, or a range of angles due to the curvature of the surface. Device angles may range between 0 and 90 degrees, but more ideally between 0 and 65 degrees. Additionally, a number of variables including materials of construction, surface finish, contact angles, hole and gap widths can be altered to further optimize manufacturing operations.

### PATENTS CITED

US 2007/0163328, Apparatus and Test Procedure for Measuring the Cohesive, Adhesive, and Frictional Properties of Bulk Granular Solids, Johanson, 2007

U.S. Pat. No. 3,940,997, Apparatus and Method for Measuring Angle of Repose, Hudson, 1976 CN109085116A, Friction Coefficient Tester and Measuring Method, Hebei University, 2018

### REFERENCES CITED

Jenike, A., Storage and Flow of Solids—Bulletin 123, University of Utah, Salt Lake City, Utah, 1964

The United States Pharmacopeia. The National Formulary. Rockville, Md. :United States Pharmacopeial Convention, Inc., 1979.

### BRIEF DESCRIPTION OF DRAWINGS

FIG. 1 is a perspective view of the recommended procedure with a fixed base for Angle of Repose in accordance with USP SECTION <1174> POWDER FLOW;

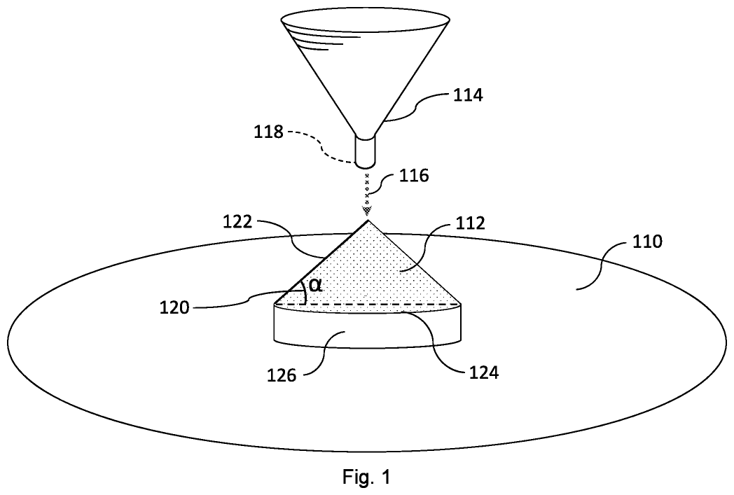

FIG. 2 is a perspective view of a single angle conical shaped surface test device according to the current invention;

FIG. 3 is an orthogonal view of a multi-angled test device according to the current invention;

FIG. 4 is an orthogonal view of a curved surface (hemisphere) test device with a granular material conical pile formation according to the current invention;

FIG. 5 is an orthogonal view of a conical pile formation of material having excellent flow properties according to the current invention;

FIG. 6 is an orthogonal view of a conical pile formation of material having fair flow properties according to the current invention;

FIG. 7 is an orthogonal view of a curved surface (modified hemisphere) according to the current invention;

FIG. 8 is a cut away view of a convex funnel design according to the current invention;

FIG. 9 is a perspective view of a single linear rail type design according to the current invention;

FIG. 10 is a perspective view of an exploded view of a dual linear rail design according to the current invention; and

FIG. 11 is a perspective view of an assembled dual linear rail type design according to the current invention.

### REFERENCE NUMERALS

110 Flat level surface used to form conical pile of granular material

112 Conical pile of granular material

114 Fixed funnel, position adjustable

116 Granular material flow

118 Gate

120 Angle of Repose of granular material

122 Upper surface of conical pile

124 Base of conical pile

126 Test device base

128 Single angle conical shaped test device

130 Upper surface, lower angle from horizontal

132 Lower surface, higher angle from horizontal

134 Transition between upper and lower angled surfaces

136 Transition between lower angled surface and test device base

138 Curved surface test device (hemisphere)

140 Optional inscribed angle indicators

142 Pile formation of granulation having excellent flow properties

144 Conical pile base, sample retention boundary

146 Angle of conical pile for granulation having excellent flow properties

148 Pile formation of granulation having fair flow properties

150 Angle of conical pile for granulation having fair flow properties

152 Curved surface of modified hemisphere

154 Start angle of modified hemisphere

156 End angle of modified hemisphere

158 Cut-away of convex funnel

160 Segment from convex funnel

162 Steep angle of convex funnel

164 Shallow angle of convex funnel

166 Sample retaining ring

168 Discharge hole

170 Left facing warped surface of linear test device

172 Inner side of linear test device

174 Outer side of linear test device

176 Rear of linear test device

178 Front of linear test device

180 Shallow angle of linear test device

182 Steep angle of linear test device

184 Right facing warped surface of linear test device

186 Base for linear test device

188 Right facing linear test device assembly

190 Left facing linear test device assembly

192 Gap of adjustable width

### DETAILED DESCRIPTION

The following examples define and describe the apparatus for the invention and methods of use thereof and are not intended to limit in any way the scope of the invention.

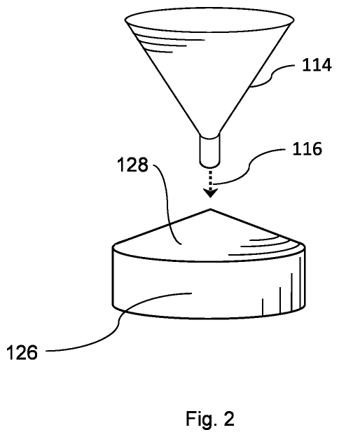

An apparatus FIG. 2 used for quality control (go/no go) applications to evaluate granular materials for conformance to specified angle of repose and flow criteria wherein a conical surface 128 forming an angle as such that only granulations exhibiting unacceptable flow properties will be retained on the device. The apparatus FIG. 2 consists of an adjustable funnel stand (not shown), a funnel with an optional gate, and a test device consisting of a base 126 and a top piece with a conical upper surface 128. The base should be made of suitable height to allow for excess material to fall to the side without interfering with the test. The base 126 and top piece 128 can also be made from a single piece of material. The conical surface is made at an angle suitable for differentiating between acceptable and unacceptable materials. Ideally, the angle of the test device surface (θ) should not exceed the highest acceptable angle of repose (α).

Alternatively, the apparatus FIG. 2 can be used to determine when the angle of repose of a sample is too low. A device consisting of a conical surface having an angle below the lowest acceptable angle of repose (α) can be utilized to indicate problematic granulations. Additionally, a sample retaining ring placed on top of the device at the outer edge is utilized to conduct static testing when lifted in a controlled manor.

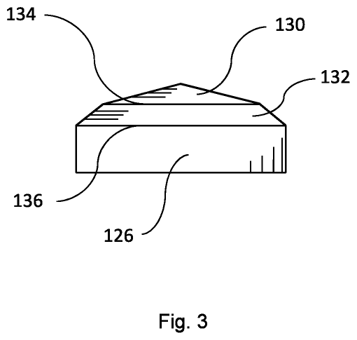

An apparatus FIG. 3 consisting of an adjustable funnel stand (not shown), a funnel with an optional gate (not shown), base 126, and a top piece having an upper surface with a dual level multi-angled conical shape used for evaluating upper and lower acceptance criteria. The example FIG. 3 consists of an upper level 130 set at a shallower angle, and a lower level 132 set at a steeper angle. The upper level starts at the top center point and extends down to the first transition 134, the point at which the low and high angles meet. The lower level starts from the first transition 134 and extends down to the second transition 136, the point at which the high angle and the side of the base 126 meet. The base 126 and top piece can optionally be manufactured from a single piece of material.

The upper level 130 is used to determine acceptance of granular materials having a lower angle of repose while the lower level 132 is used to determine acceptance of granular materials having a higher angle of repose. Granular test samples are poured onto the top center of the device. Samples having a higher angle of repose (α) than the test device angle will be retained. Samples having a lower angle of repose (α) than the test device angle will slip off to the sides of the device. A base of sufficient height 128 is required to prevent excess sample from building up and interfering with the test. A sample retaining ring can also be placed (not shown) on top of the device at the outer edge of the sphere. This can be utilized to provide static testing when lifted in a controlled manor.

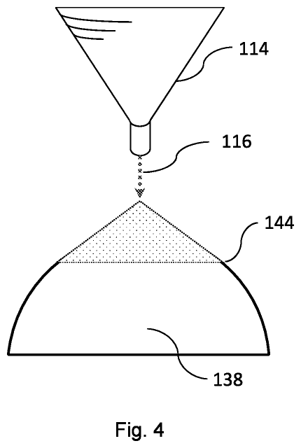

An apparatus FIG. 4 consisting of an adjustable funnel stand (not shown), a funnel with an optional gate, and a curved surface test device 138. The funnel 114 and test device 138 are ideally aligned along a central axis whereas the granulation 116 is uniformly distributed across the entire surface. The test device can optionally be placed on a base to provide additional height should larger amounts of material be required.

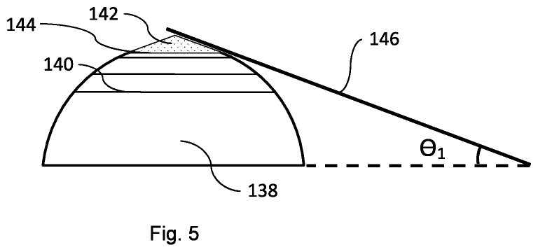

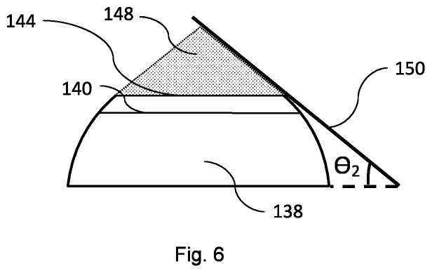

An apparatus FIG. 5 and FIG. 6 consisting of an adjustable funnel stand (not shown), a funnel with an optional gate (not shown), a test device with a curved surface in the shape of a hemisphere 138 with lines 140 inscribed at positions representing various angles along the curved surface. This method provides several advantages over methods described in the prior art as it is not necessary to measure the pile with a height gage, or measure the angle with a protractor, nor is it necessary to assemble a perfectly formed cone to obtain an accurate reading as this method only requires noting the position of the sample retention boundary 144.

The example in FIG. 5 shows a conical pile of granular material having excellent flow properties 142. A smaller amount of material will be retained on the device resulting in a higher position of the sample retention boundary 144. A line 146 drawn tangent to the sample retention boundary 144 point of contact shows that Aᵢ is relatively small.

The example in FIG. 6 shows a conical pile of granular material having fair flow properties 146. A larger amount of material will be retained on the device resulting in a lower position of the sample retention boundary 144. A line 150 drawn tangent to the sample retention boundary 144 point of contact shows that θ₂ is larger.

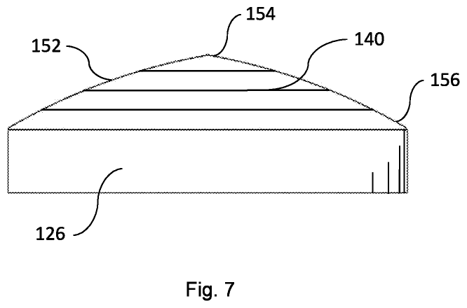

An apparatus FIG. 7 consisting of an adjustable funnel stand (not shown), a funnel with an optional gate (not shown), a base 126, and a top piece 152 resembling a modified hemisphere. The slope nearest the center 154 has a lower angle as compared to the slope of the surface nearest the outer edge 156. The starting angle 154 and ending angle 156 are determined based upon the characteristics of the powders or granular materials to be evaluated. A higher resolution per surface area within a range of angles is provided with this modification as angles of lesser interest are not present.

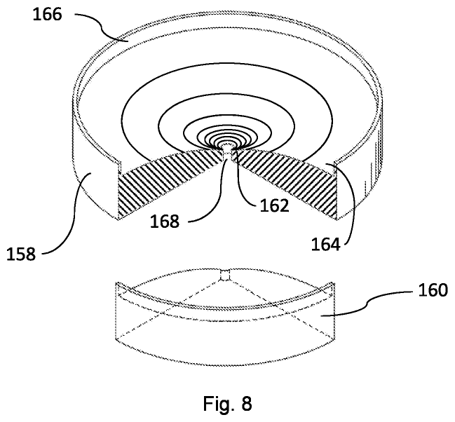

An alternative design apparatus FIG. 8 consisting of an adjustable funnel stand, a funnel with an optional gate, an optional base (not shown), and a top piece having a surface resembling a convex shaped funnel 158. The slope of the surface nearest the outer edge 164 includes a lower angle compared to the slope of the surface nearest the center 162. A plug or rod is utilized to close off the hole during sample filling. A discharge hole 168 located in the center is included for removal of excess and non-retained material. A sample retention ring 166 is shown in the attached configuration which aids with filling the device, prevents spillage, and allows for deeper sample packing. A stand of suitable height with a hollow center should be utilized to aid in removing excess material. The starting angle 164, ending angle 162, and exit hole 168 are determined based upon the characteristics of the powders or granular materials to be evaluated. A higher resolution per surface area within a range of angles is provided with this modification as angles of lesser interest are not present.

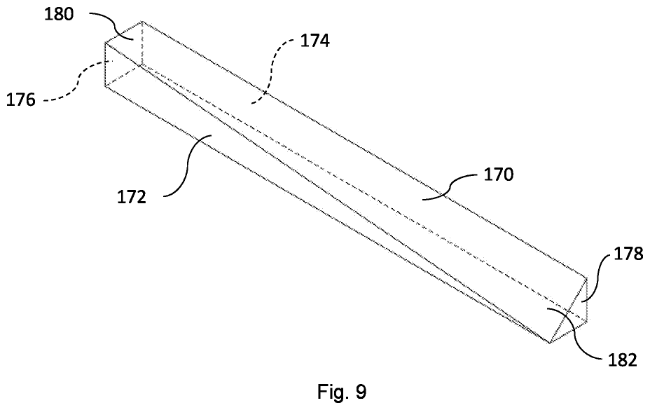

Another alternative design apparatus FIG. 9 consisting of an adjustable funnel stand, a funnel or hopper suitable of dispensing materials along a linear path, an optional base (not shown), and a bar shaped top piece 170 whereas the upper surface consists of a range of angles along its length. The upper surface at the furthest end 176 has a shallower angle 180 than that of the nearest end 178 having a steeper angle 182. Angles along the length of the bar are formed by altering the height of the inner side 172 while maintaining the height of the outer side 174. The angular rate of change from low 180 to high 182 as shown in FIG. 9 is linear but can optionally be non-linear. As with designs previously described in the invention, granulated materials will be retained at lower angles up to the point the sample retention boundary. Granulations above this point will not be retained and flow will occur from the direction of the outer side 174 towards the inner side 172. The advantage of this design is that surface area for all angles is constant as the highest point is not reduced to a single termination point as with circular designs. An additional advantage to this design is that variability when applied free hand is reduced, making it more suitable for use with potent and toxic compound work within an isolator.

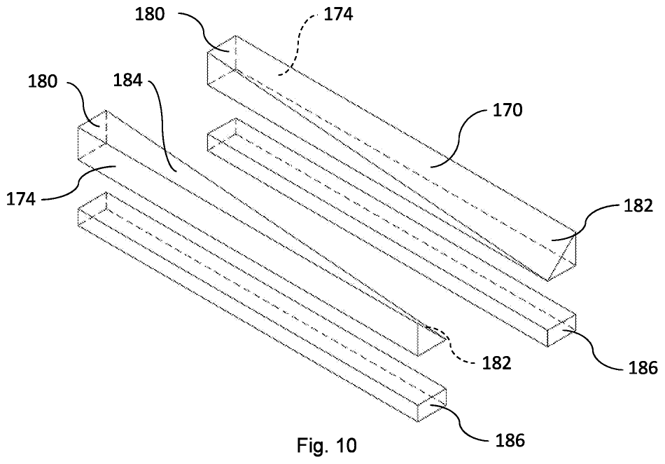

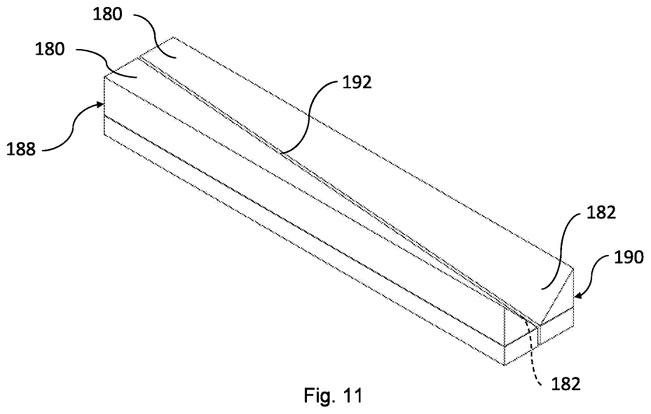

An alternative configuration FIG. 10 and FIG. 11 consists of two linear style test devices as describe above but with opposing slope directions. A right facing assembly 188 is combined with a left facing assembly 190 and connected with a spacer between to provide gap width 192 adjustments between the assemblies 188 and 190. two bases 186 are utilized to allow for removal of excess material during testing.

The configuration described above creates a funnel FIG. 11 having a range of feed angles along its length and can be used to aid in hopper and process equipment evaluations or to characterize granulations for bridging or ratholing issues.

An alternative configuration (not shown) consists of two linear style test devices as describe above manufactured with opposing slopes and placed with inner sides together. A right facing assembly having a fixed angle is combined with a left facing assembly having the same angle and connected with a spacer between to provide gap width adjustments. Variations include, but are not limited to the following:

Adjustable gap width along the length of the assembly.

Adjustable funnel height along the length of the assembly.

Variable surface finish along the length of the assembly.

To characterize granular material 116 with use of test devices described in FIG. 4-FIG. 8, an amount is added to the test device 138 until a conical pile of maximum size is obtained. Upon initiation of the test the granulation will initially be retained on the upper most section of the test device as this is nearest to level. As more material is applied a larger pile is formed, spreading out over the device to increasingly steeper portions. Material will be retained until it reaches the “sample retention boundary” 144, the point in which material can no longer be supported. The position of this boundary occurs at lower angles for granulations having excellent flow properties while at higher angles for those with poor flow properties. The benefit of the invention is that the sample retention boundary can be quickly determined.

A small difference in the position of the sample retention boundary 144 and the point tangent to the angle of repose have been observed. This difference is due in part to the lack of an underlying foundation required to initiate the formation of a granular pile at angles approaching that of the repose angle. The characterization of this difference should be useful in further determining process equipment design requirements.

What is claimed is:

1. An apparatus for measuring flow properties of granular materials comprising: a means of supporting the test device; and
a test device consisting of an upper surface with a smooth finish and set at one or more angles wherein a sample retention boundary is defined.

2. The apparatus of claim 1, wherein the test device upper surface has a centrally located high point and equally decreases in height in the radial direction.

3. The apparatus of claim 1, wherein the test device upper surface has a non-centrally located high point and decreases in height in the radial direction.

4. The apparatus of claim 1, wherein the test device upper surface has a high point along a non-linear path and decreases in height at an angle perpendicular to the non-linearpath.

5. The apparatus of claim 1, wherein the test device upper surface has a high point along a non-linear path located about the outer radius and decreases in height at an angle perpendicular to the non-linear path and a hole is centrally located on the lower surface to allow for discharge of test materials.

6. The apparatus of claim 1, wherein the test device having a length greater than its width and wherein the upper surface has a high point along its length and decreases in height along its width.

7. The apparatus of claim 1, wherein the test device having a length greater than its width and wherein the upper surface has a high point along its length at the midpoint of the width and decreases in height in opposing directions along its width.

8. The apparatus of claim 1, wherein the test device having a length greater than its width and the upper surface has a high point along its width at one end wherein the height is constant along its length along the intersection of the upper surface and the left side and wherein the height decreases in height at a constant rate along the intersection of the upper surface and the right side.

9. The apparatus of claim 1, wherein the test device having a length greater than its width and the upper surface has a high point along its width at one end wherein the height is constant along its length along the intersection of the upper surface and the right side and wherein the height decreases in height at a constant rate along the intersection of the upper surface and the left side.

10. The apparatus of claim 1, wherein the test device upper surface has a polished surface finish.

11. The apparatus of claim 1, wherein the test device upper surfacehas a roughened surface finish.

12. The apparatus of claim 1, wherein the test device upper surface has a grooved surface finish.

13. An apparatus for measuring flow properties of granular materials comprising:
a test device consisting of an upper surface with a smooth finish and set at one or more left facing angles wherein a sample retention boundary is defined.
a test device consisting of an upper surface with a smooth finish and set at one or more right facing angles wherein a sample retention boundary is defined.
a means of supporting the test devices; a
means of joining the test devices; and
a means of forming a gap and adjusting the width between the test devices.

14. The apparatus of claim 13, wherein both the test devices having a length greater than its width and wherein the upper surface has a high point along its length and decreases in height along its width.

15. The apparatus of claim 13, wherein the test device having a length greater than its width and the upper surface has a high point along its width at one end wherein the height is constant along its length along the intersection of the upper surface and one side and wherein the height decreases in height at a constant rate along the intersection of the upper surface and the opposing side.

16. The apparatus of claim 13, wherein the test device upper surface has a polished surface finish.

17. The apparatus of claim 13, wherein the test device upper surface has a roughened surface finish.

18. The apparatus of claim 13, wherein the test device upper surface has a grooved surface finish.

19. A method of testing granular material using the apparatus of claim 1, wherein the granular test material is poured onto the highest point of the upper surface of the test device by means of a fixed funnel having adjustments to minimize the height of the drop of the granular material. The test is completed once the size of the pile formed on the device reaches its maximum size and the point in which material is no longer retained is noted. This result is read from a scale inscribed on the surface of the test device or by other means.

20. A method of testing granular material using the apparatus of claim 13, wherein the width of the gap is adjusted and wherein the granular test material is poured into the midpoint along the length of the device by means of a funnel having adjustments to minimize the height of the drop of the granular material.
